(19) United States
(12) Patent Application Publication
(10) Pub. No.: US 2026/0345046 A1
(43) Pub. Date: Nov. 5, 2026

(54) Biohybrid Medical Device

(71) Applicant: CAMBRIDGE ENTERPRISE LIMITED, Cambridge (GB)

(72) Inventors: George Malliaras, Cambridge (GB); Damiano Giuseppe Barone, Houston, TX (US); Alejandro Carnicer Lombarte, Cambridge (GB); Mark Kotter, Cambridge (GB); Malak Kawan, London (GB)

(21) Appl. No.: 19/142,443

(22) PCT Filed: Dec. 21, 2023
(86) PCT No.: PCT/EP2023/087417

§ 371 Date: Jun. 23, 2025

(30) Foreign Application Priority Data
Dec. 23, 2022 (EP) 22386099.0

(51) Int. Cl. A61B 5/268; A61B 5/00

(52) U.S. Cl. CPC A61B 5/268; A61B 5/4041; A61B 5/407; A61B 5/4836; A61B 5/6868; A61B 5/6877

(57) ABSTRACT

A medical device, comprising a flexible electrode array having a bend radius of no more than about 2 mm; and a culture of biological cells on the flexible electrode array and integrated with the electrodes of the flexible electrode array.

### FIELD

The present invention relates to a medical device having an electrode array. The invention is of particular relevance to implantable devices, for example those interfacing with biological tissue such as the nervous system for purposes such as controlling prosthetic limbs, recording cellular activity for scientific or diagnostic purposes, electrical stimulation, pain management, rehabilitation, and brain-machine interfaces more generally.

### BACKGROUND

Neurological disability affects over 1.3 billion people worldwide. Brain, spinal cord and peripheral nerve injuries result in a disconnection in the nervous system communication and a loss in neurological function. Nowadays, there is no cure for these debilitating conditions. Brain-machine interface and neuroprosthetic technologies are an approach to restore lost neurological function; as in theory electronic connections can be created directly from one part of the nervous system to another, bypassing the site of injury. However, current implantable electrodes have shown limited efficacy and lifetime.

A major hurdle in reversing the effect of injury to the peripheral nervous system is the inherent inability of neurons to regenerate and to re-build disrupted neural circuits. Implantable neurotechnology and cell therapy are developing as potential effective treatments. These methods attempt to restore function by either bypassing the injury site and electrically interacting with existing neurons or providing new cells to replace the damaged ones. Unfortunately, they both have drawbacks, which have slowed down their translation to the clinic. In the context of damaged tissue in the mature nervous system, transplanted neurons struggle to re-establish functional connections in existing circuits without appropriate guidance. Similarly, electrodes cannot work without healthy working cells to interface, either because these cells are compromised by the injury or hidden by the formation of dense scar tissue around the implant (i.e., foreign body reaction (FBR)). Moreover, current neurotechnologies lack the selectivity and specificity to interface to different subtypes of neurons responsible for different functions.

### SUMMARY

The invention results from a combinational approach of electronics and cell transplantation for functional neurological restoration.

The invention aims to improve the integration between implantable electronics and existing tissue. The invention, thanks to the incorporation of cells, allows for synaptic integration between implanted cells and flexible circuitry. The benefits of this strategy are: an ability to host and interact with stem-cell derived cells (in vitro); promote functional cellular integration with living tissue (in vivo); and regain lost neurological function.

There is disclosed a multielectrode array (MEA) neural interface design. The device preferably hosts the cells in an electrically-active conduit that provides efficient electrical stimulation. The activation of the muscle can take place through a neuromuscular junction, addressing the problems of efficiency and stability that plague current devices. The conduits on flexible substrates can be fabricated using microfabrication techniques, comprising conducting polymer electrodes that combine topographical guidance and electrical stimulation to guide reinnervation.

Personalised electronic therapies are being developed to include biologically-inspired materials to treat nervous system injuries. One limiting factor is the resolution with which nerve inputs can be mapped onto implants. A biohybrid strategy incorporating cells as an intermediate layer on the electronics allows for a ‘controllable’ synaptic integration between implanted cells and existing neural circuitry. Biohybrid implants thus have the potential ability to host, interact and control the behaviour of transplanted cells; promote organised, functional cellular integration with living tissue and reduce scar tissue formation (i.e., FBR).

The use of a scalable cell source, which can be integrated into a bioelectronic device as a biological target for peripheral nerve inputs, may increase recording resolution.

There is thus disclosed a novel biohybrid neural interface, combining long-term surviving iPSC-derived human skeletal myocytes and flexible electronics. The device was validated in a chronic sensorimotor nerve rat model, and demonstrated tissue integration and enhanced electrophysiology recordings.

There is disclosed a medical device, comprising a flexible electrode array having a bend radius of no more than about 2 mm; and a culture of biological cells on the flexible electrode array and electrically integrated with the electrodes of the flexible electrode array.

The electrical integration of the cells with the electrodes allows a connected device to read signals derived from the connected tissue, and also allows a connected device to supply signals, e.g. motor signals, to the tissue. The invention thus opens the way in the field of prosthetic limbs.

With regard to applications in prosthetics, prior products rely on the recording of signals from existing muscles in patients. This means repurposing some existing muscles to guide a prosthetic arm via electrodes. The present biohybrid device does record from cells, but the cells (e.g. muscle cells) used are implanted, and their function is to be part of the implant and integrate with the host nerve.

Unlike existing myoelectric prosthetics, existing musculature is not repurposed to drive a prosthesis. This means that the resolution of a muscle is no longer a limiting factor (where a number of patterns of contraction being recorded will usually be quite low, particularly for systems with a few electrodes sitting outside of the muscle).

According to the method of the invention, the (e.g. muscle) cells are grown directly on the implant itself in order to have independent access to very small groups of muscle cells. Once these are innervated by the host nerve, this allows many more degrees of freedom than known systems, and so much finer control can be achieved.

The culture of biological cells optionally comprises at least one of myocytes, neurons and fibroblasts.

The use of myocytes, e.g. skeletal myocytes allows good connection with in vivo tissue to be established. By switching out muscle cells for other cell types suitable for other tissues, a biohybrid system can be made to interface with any number of tissues. Using neurons that grow out of the device, for example, one can interface with the retina or with the cochlear nerve to produce novel prostheses. Other applications are brain and spinal cord implants, to achieve better electrical stimulation in DBS/spinal cord stimulation, for example.

The biological cells are optionally generated from human induced pluripotent stem cells (iPSCs).

The culture of biological cells of the medical device may be, as opposed to a culture of entire cells, a plurality of biological cell components. That is, the medical device optionally comprises a plurality of biological cell components on the flexible electrode array and electrically integrated with the electrodes of the flexible electrode array. The plurality of biological cell components may be cell membranes, such as neuronal membranes, for example human iPSC-derived neuronal membranes.

The flexible electrode array optionally comprises electrodes provided on a flexible substrate.

The flexible substrate optionally comprises at least one of a parylene sheet and a polymer sheet comprising parylene.

The flexible electrode array optionally comprises conducting polymer electrodes.

The conducting polymer electrodes optionally comprise PEDOT:PSS.

There is further disclosed a method of making a medical device, said method comprising providing a flexible electrode array having a bend radius of no more than about 2 mm; and culturing biological cells on the flexible electrode array so that they become electrically integrated with the electrodes of the flexible electrode array.

The biological cells are optionally cultured on a layer comprising at least one of fibrin, hydrogel and non-conducting coating agent.

The biological cells optionally comprise at least one of myocytes, neurons and fibroblasts.

The biological cells optionally comprise skeletal myocytes.

The biological cells are optionally generated from human induced pluripotent stem cells (iPSCs).

The step of culturing biological cells on the flexible electrode array may be, as opposed culturing entire biological cells, depositing biological cell components on the flexible electrode array. That is, the method optionally comprises depositing biological cell components on the flexible electrode array so that they become electrically integrated with the electrodes of the flexible electrode array. The biological cell components may be cell membranes, such as neuronal membranes, for example human iPSC-derived neuronal membranes.

There is disclosed a method of implanting a medical device as described herein in a subject, the method comprising implanting the device and suturing the cell-laden side of the device to tissue in the subject.

The cell-laden side of the device is optionally sutured to tissue comprising at least one of a nerve, a muscle, skin, brain or spinal cord to facilitate electrical communication between said tissue and said device.

The method optionally further comprises anchoring the device in the subject by suturing the device to anchoring tissue of the subject.

There is disclosed a method of treating a human or animal body, the method comprising implanting a medical device as described herein.

The electrode array is flexible, having a bend radius of no more than 2 mm, preferably no more than 1.5 mm and more preferably no more than 1 mm. Bend radius, which is measured to the inside curvature, is the minimum radius that a component (in this case the electrode array) can be bent in at least one direction without damaging it. The bend radius as defined here refers to elastic deformation as opposed to plastic deformation such that an electrode array bent under an applied force to a radius greater than the minimum bend radius would return at least part way to its original shape with the removal of the applied force. In other words, the electrode array in the device can be bent to an inside curvature of 2 mm, for example by rolling (e.g. when the device is being arranged for insertion into a patient), and subsequently deployed (e.g. unrolled) to an expanded, less bent configuration (e.g. a substantially planar configuration) and still function exactly as it did prior to bending.

Preferably the flexible electrode array has a bend radius of no more than about 1.5 mm, more preferably no more than about 1 mm, more preferably no more than about 0.5 mm.

The electrode array is based on a substrate which could for instance take the form of thin layers of parylene-C or polyimide with or without layers of silicone.

The electrode array may be provided with openings, e.g. circular opening, to permit growth of vasculature from the back of the device and support cell survival post-implantation.

The distal section of the device may contain the flexible and re-configurable components such as the electrode array, whilst less flexible (or inflexible) components such as connectors can be located in the proximal section.

The terms distal section and proximal section are intended to refer to the relative arrangement of the components described in this aspect. In particular, in certain embodiments, it is not envisaged that the device itself includes any wires or other connectors (e.g. tubes) which serve to connect the device to further apparatus or devices (such as controllers and/or fluid and/or power sources) external to, or at the skin level of the subject after insertion of the device. Thus the proximal section of the device may solely contain the components necessary to make connections to such items.

In such arrangements, the proximal section of the device may form a relatively small proportion of the device as a whole, for example no more than 20%, preferably no more than 15%, more preferably no more than 10%, more preferably no more than 5% of the total volume of the device in the deployed state (such that the distal section having the active components comprises 80%, 85%, 90% or 95% of the volume of the device respectively).

The device may further comprise a conductive connector connecting the electrode array to an electrical connector.

Optionally, the medical device is a bioelectric implant. The bioelectric implant may be an active implant, such as a spinal cord or nerve stimulator. The bioelectric implant can be a passive implant, such as an electrocorticography sensor or other sensor.

Optionally, the flexible electrode array comprises electrodes provided on a flexible substrate. The flexible substrate may be 500 μm thick or less, optionally 200 μm thick or less, further optionally 100 μm thick or less, further optionally 50 μm thick or less, further optionally 25 μm thick or less, further optionally 10 μm thick or less and still further optionally 5 μm thick or less. The flexible substrate may be made of a polymeric material, optionally a thermoplastic, and optionally comprising one or more of a poly-urethane, a silicone, a parylene, a polyimide, a polyamide, a cyclic olefin polymer, a cyclic olefin copolymer, a polyacrylate, polyethylene terephthalate and/or an epoxy. The preferred material for the substrate is parylene C. Polydimethylsiloxane (PDMS) may also be used, either alone or in combination with parylene.

The medical device may include one or more components which are imageable by X-ray such as a strip of a polymer material infused with BaSO₄. This allows the position of the device to be checked and/or monitored during and/or after the device has been deployed in a subject.

According to a further aspect of the invention, there is provided a method of treating a human or animal body, the method comprising implanting a medical device or bioelectric implant according to any of the variations of the device disclosed herein.

There is further disclosed a medical device, comprising a flexible electrode array having a bend radius of no more than about 2 mm; and a plurality of biological cell components on the flexible electrode array and electrically integrated with the electrodes of the flexible electrode array. The biological cell components may be cell membranes, such as neuronal membranes, for example human iPSC-derived neuronal membranes.

There is further disclosed a method of making a medical device, said method comprising providing a flexible electrode array having a bend radius of no more than about 2 mm; depositing biological cell components on the flexible electrode array so that they become electrically integrated with the electrodes of the flexible electrode array. The biological cell components may be cell membranes, such as neuronal membranes, for example human iPSC-derived neuronal membranes.

### BRIEF DESCRIPTION OF THE FIGURES

The invention is described below, by way of example, with reference to the accompanying figures in which:

FIG. 1 is a perspective view of a medical device as disclosed herein.

FIG. 2 is a schematic drawing showing a step-by-step process involved in making the medical device.

FIGS. 3A-3C show the medical device in the form of a biohybrid peripheral neural interface, in which:

FIG. 3A shows an in vivo biohybrid device design customised for the peripheral nervous system (Scale bar: 60 μm),

FIG. 3B is a brightfield image showing human iPSC derived myocytes at Day 8 of culture (Scale bar: 465 μm) and

FIG. 3C is a brightfield image showing human iPSC derived myocytes at Day 8 of culture (Scale bar: 230 μm).

FIG. 3D shows a completed biohybrid device, including a fibrin hydrogel, and iPSC derived muscle cells (Scale bar: 1 cm).

FIG. 3E shows the electrode impedance at 1000 Hz before and after cell addition over a 4 week period post implantation.

FIG. 3F shows an experimental timeline showing the fabrication and implantation of the biohybrid device from an in vitro cell culture step into an animal model.

FIG. 4A shows the surgical implantation of the biohybrid device into the rat forearm nerve bundle (Scale bar: 2 mm).

FIG. 4B shows an image of human nucleoli stained (red/brown) biohybrid device after 28 days of implantation where the survival of a layer of human iPSC derived muscle cells visible as a layer of stain near the parylene of the device (Scale bar: 50 μm).

FIG. 4C shows magnified images of control device (i.e. a device lacking iPSC cells) and biohybrid implants 28 days post-implantation.

FIG. 4D shows human nucleoli stain intensity (ratio to background) over distance from the implant in both the control (black) and biohybrid device implants (red) (mean±SD).

FIG. 4E shows average human nucleoli stain intensity in the 10 μm layer closest to the implant.

FIG. 5A shows a schematic of the experimental setup.

FIG. 5B shows representative traces of nerve response to 100 μA, 0.1 ms stimulation pulses, taken from different electrodes within a device MEA.

FIG. 5C shows average pk-pk CAP amplitude from the MEA of a the biohybrid device from FIG. 5B.

FIG. 5D shows quantification of pk-pk CAP amplitude in 28 day-implanted biohybrid and control devices for 100 μA pulse stimulation.

FIG. 6A is a photograph of an experimental setup.

FIGS. 6B-6F show how nerve electrical recordings from biohybrid devices progressively improve over four weeks post-implantation, coinciding with nerve regeneration.

FIG. 6B shows forearm nerve signal traces recorded from biohybrid devices over four weeks post-implantation.

FIG. 6C shows quantification of signal-to-noise ratio (SNR) of recorded traces.

FIG. 6D shows RMS time traces from three bipolar electrodes (cyan, green, magenta) over four weeks of implantation, normalised to range from 0 to 1 for each week.

FIG. 6E shows quantification of correlation between recorded activity and stepping events with implanted paw.

FIG. 6F shows sample time-frequency spectrogram and time trace of a recording from a bipolar electrode at week 4 post-implantation.

FIGS. 7A-7C show the stages of development of neuronal membrane-derived biohybrid bioelectronics (NMemb-Biohybrids).

FIG. 7A shows blebbing of human iPSC-derived glutamatergic neurons at day 8 of cell-culturing.

FIG. 7B shows the subsequent fusion of the neuronal-derived vesicles into SLBs (using fusogenic liposomes composed of a DOPC/DOTAP mixture and PEG) encapsulated within fibrin hydrogels on bioelectronic devices.

FIG. 7C shows an in vivo bioelectronic device (using flexible ECoG arrays on Parylene C (PaC) substrates) design customized for subdural implantation into animal models.

FIGS. 8A-8I show optical and electrical characterization of neuronal-derived membranes on Au/PEDOT:PSS microelectrodes.

FIG. 8A shows cultured neurons undergoing blebbing under a 20× magnified, grayscale brightfield image, demonstrating the budding and vesiculation of their membranes after 2.5 hour incubation in the vesiculation buffer.

FIG. 8B shows TEM micrographs of the collected lipid vesicles.

FIG. 8C shows a concentration vs. size plot for a polydisperse solution of collected lipid vesicles measured using an NTA.

FIG. 8D shows FRAP measurements after deposition of fluorescently labelled neuronal-derived lipid vesicles (0.36 mM R18 dye) on PEDOT:PSS films after their adsorption, rupture and subsequent fusion via self-assembly with the aid of fusogenic liposomes (DOPC/DOTAP mixture, DOPC: 1,2-Dioleoyl-sn-glycero-3-phosphocholine, DOTAP: Dioleoyl-3-trimethylammonium propane) and PEG.

FIG. 8E shows Au/PEDOT:PSS microelectrodes consisting of varying geometries, including 200 μm×200 μm electrodes, which was used as the most suitable dimensions to sense these membranes. Scale bar=100 μm.

FIG. 8F shows EEC modelling of the membrane used to extract membrane resistance from EIS spectra.

FIG. 8G shows a Bode plot of impedance vs. frequency of the Au/PEDOT:PSS response to membrane depositions.

FIG. 8H shows a Bode plot of phase shift vs. frequency of the Au/PEDOT:PSS response to membrane depositions.

FIG. 8I shows a Nyquist plot of the deposited neuronal membranes, with complex imaginary (Z″) versus real (Z′) impedances being plotted (n=5).

FIGS. 9A-9E show a comparison of the electrochemical properties of native and synthetic membranes on Au/PEDOT:PSS microelectrodes.

FIG. 9A shows a schematic demonstrating the increasing complexity of SLBs: “Lipid-only” membranes assembled via vesicle fusion of unilamellar synthetic vesicles vs. complex native membranes that are derived from cellular blebs (simplified image). It displays the varying sealing properties of these SLBs once self-assembled on Au/PEDOT:PSS microelectrodes with dimensions of 200 μm×200 μm.

FIG. 9B shows a Bode plot of bare electrodes (PEDOT:PSS; black line), neuronal (purple line) and astrocyte-derived SLBs (pink line) and DOPC/DOTAP SLBs (green line), showing impedance as a function of frequency.

FIG. 9C shows a Bode plot of bare electrodes (PEDOT:PSS; black line), neuronal (purple line) and astrocyte-derived SLBs (pink line) and DOPC/DOTAP SLBs (green line), showing phase shift as a function of frequency.

FIG. 9D shows a Nyquist plot of bare electrodes (PEDOT:PSS; black), neuronal (purple) and astrocyte-derived SLBs (pink) and DOPC/DOTAP SLBs (green).

FIG. 9E shows normalized impedance for each SLB type.

FIGS. 10A-10M show a reduced FBR using neuronal membrane-derived bioelectronics in vivo following a 28 day chronic rat study.

FIG. 10A shows a timeline of cell culturing iPSC-derived glutamatergic neurons for 8 days, the cells underwent blebbing and vesicles were collected to assemble the neuronal membranes on implants encapsulated with fibrin hydrogel. At day 0, lipidized implants (PEDOT:PSS-coated) and control groups were subdurally implanted for 28 days. Brains were then extracted for histology and immunohistochemical analysis of GFAP and CD11b/c to assess FBR to neural implants.

FIG. 10B shows immunohistochemical analysis of FBR capsule (GFAP) after implanting the NMemb-Biohybrids at day 28 with stain intensity profiles of GFAP at 400 μm thickness from the edge of the cortex in which the implant is interfaced, analyzing four groups: lipidized implants, control implants (−SLB), PET implants (positive control) and sham surgery group.

FIG. 10C shows averages of the stain intensity of GFAP of the first 100 μm layer closest to the implant.

FIG. 10D shows stain intensity profiles of CD11b/c at 400 μm thickness from the edge of the cortex at the site of implantation.

FIG. 10E shows averages of the stain intensity of CD11b/c of the first 100 μm layer closest to the implant.

FIGS. 10F-10I show immunofluorescent images after extracting the brains at day 28, cryosectioning, and staining with GFAP, CD11b/c, and DAPI to observe the effects of FBR and inflammation degree to the biohybrid model at the implant site. Animals received lipidized implant. These images represent a single animal. Scale bar=250 μm.

FIGS. 10J-10M show immunofluorescent images after extracting the brains at day 28, cryosectioning, and staining with GFAP, CD11b/c, and DAPI to observe the effects of FBR and inflammation degree to the biohybrid model at the implant site. Animals received control implant. These images represent the same single animal as FIGS. 10F-10I. Scale bar=250 μm.

FIGS. 11A-11E show electrophysiological recordings of neuronal membrane-derived ECoG implants in vivo.

FIG. 11A shows an image of the device before implantation with a PDMS (Polydimethylsiloxane) well where SLBs and fibrin hydrogel are contained and interfaced on Au/PEDOT:PSS electrodes (top) and ECoG device on PaC substrate with Au-PEDOT:PSS-coated microelectrodes (a total of 32 electrode per device) used for recording LFPs (subdurally) connected to an external cable (brown cable) (bottom).

FIG. 11B shows images of fluorescently labelled membranes (0.25% BODIPY DHPE) on the electronic device pre-implantation where one electrode (pink box) demonstrates mobility of the membrane after photobleaching the defined ROI (dark circle) on the site with a dimeter of 30 μm. Images on the right display photobleaching at t=0-40 seconds, demonstrating the recovery in fluorescent intensity overtime, and thus lateral diffusion of the lipid molecules on the ECoG surface. Scale bar=30 μm.

FIG. 11C shows a schematic of the subdural implantation or brain target, showing both lateral parietal areas between Bregma and Lambda where the implant is interfaced.

FIG. 11D shows LFPs measured using two different arrays (control vs. lipidized implants), demonstrating that electrophysiological recordings are successful with the lipidized implant. Note: the devices are recording different areas, hence, showing different LFP traces, N=2, n=4.

FIG. 11E shows a captured image of the subdural implantation surgery, showing PEDOT:PSS-based ECoG arrays: one encompassing the neuronal membranes within a fibrin hydrogel, while the other does not. LFP=local-field potential.

### DETAILED DESCRIPTION

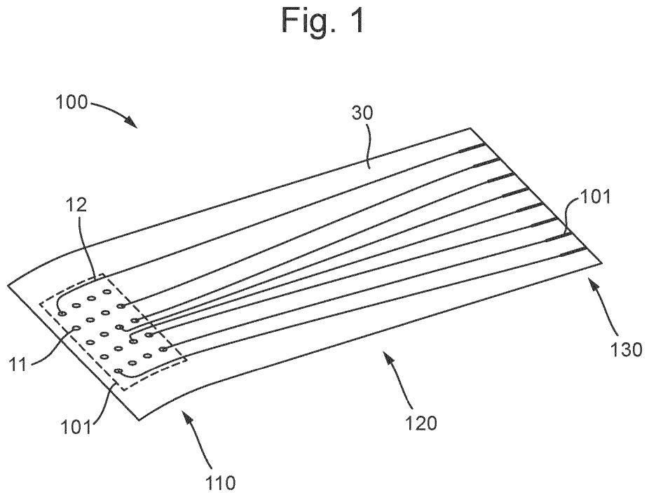

FIG. 1 illustrates a medical device 100. The medical 100 may be a bioelectric implant, for example. The bioelectric implant 100 may be an active implant, such as a spinal cord stimulation (SCS) device. Alternatively, the bioelectric implant may be a passive implant, such as an electrocorticography sensor. In other applications, device 100 may have both active and passive functions. Other applications for such devices 100 include for use in peripheral nerve implants or recording/stimulating muscle activity.

The medical device 100 comprises a flexible electrode array 10. The flexible electrode array 10 comprises electrodes 11 connected to conductive lines 12, provided on a flexible substrate 30. By way of non-limiting example, the flexible electrode array may be around 5 μm thick.

The flexible substrate 30 may be 500 μm thick or less, optionally 200 μm thick or less, further optionally 100 μm thick or less, further optionally 50 μm thick or less, further optionally 25 μm thick or less, further optionally 10 μm thick or less and still further optionally 5 μm thick or less. A thin substrate facilitates the incorporation of the medical device 100 into biological tissue.

The flexible substrate 30 may be made of a polymeric material, optionally a thermoplastic, and optionally comprising one or more of a poly-urethane, a silicone, a parylene, a polyamide, a polyimide, a cyclic olefin polymer, a cyclic olefin copolymer, a polyacrylate, polyethylene terephthalate and/or an epoxy. Such materials are suitable for implantation in the body, and provide the desired flexibility.

The device may have a first, distal, section 110 and a proximal section 130. There may be an intermediate section located therebetween.

The second, more proximal, section 130 may provide one or more electrical connectors 101 for electrical connection of the electrode array 10 to external electronics such as a pulse generator for stimulation or sensors for recording data from the electrodes. The electrical connectors 101 can be copper/polyimide flex cables each with a thickness of about 0.07 mm.

This distal end 110 is generally flexible, whilst the proximal and intermediate sections 120, 130 at the proximal end of the device may be less flexible or even rigid, thereby allowing for secure connection from the external sources to the fluidic component 20 and the electrode array 10. It will be appreciated that the electrical connectors 101 will likely not connect directly to the external sources but may be connected to further elements such as tubing or wires (not shown) which extend away from the medical device 100 and, when the device 100 is deployed within a subject, may extend outside of the patient through a lumen.

The distal end 110 of the device 100, and in particular the electrode array 10, has a bend radius of no more than 2 mm in the x-direction.

Having discussed the configuration of the device 100, the following sections consider options for fabrication of such a device 100. Although these protocols refer to specific manufacturing techniques, the skilled person will understand that other techniques may be substitutable to produce the devices, depending on the desired materials etc. Such processes include for example photolithography process, casted elastomer processes, digital manufacturing processes (controlled extrusion, additive manufacture).

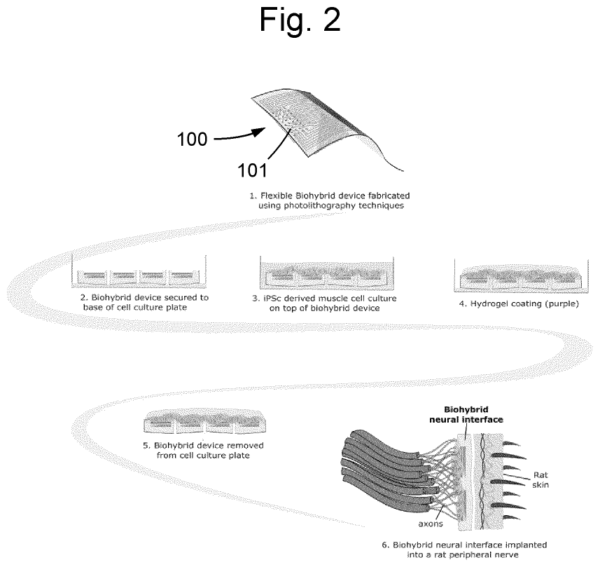

FIG. 2 is a schematic drawing showing a step-by-step process involved in making the medical device, involving culturing human iPSC derived muscle onto biohybrid neural interfaces. First, in general terms, thin film devices are fabricated using photolithography techniques. These devices are then temporarily adhered to cell culture plates for human iPSC derived muscle culture. Once the muscle cells are mature, a fibrin hydrogel is polymerised on top of the device and cells to ensure they are not damaged during surgical implantation. The device is then removed from the cell culture plate and implanted into the subject, preferably to interface with a nerve of the subject.

The described biohybrid device offers a number of advantages. The use of cell types which naturally perform signal amplification (such as skeletal myocytes, which produce a high amplitude electrical signal in response to a lower amplitude neuronal signal mediated via a neuromuscular junction) and which are placed in high proximity to the electrodes (being directly cultured on them) enables very high amplitude signal recordings. High signal-to-noise ratio is valuable in a medical device designed to record electrical signals with a neural origin, given their naturally low amplitude at their source.

The control over cell types which integrate, potentially through specific synapse formation, with host tissue, also enables novel, highly advantageous selectivity in signal recording. Electrical recordings through medical devices often measure signals from all sources indiscriminately. Often, some of the signals come from targets (e.g., neurons, neuronal populations, nerves) which are desirable to drive a particular application, while the remaining are not useful and therefore contribute to the recorded noise, occluding the signal of interest. As tissue integration is an active process, driven by, for example, chemoattractant release and membrane protein recognition at the biohybrid cell layer, certain populations in the target tissue can be selectively integrated to. As integrated populations produce highly amplified signals in the biohybrid device, as described above, this allows selective recording from desirable tissue populations, enabling further effective amplification of the signal of interest and enabling new applications requiring recording of otherwise inaccessible populations. As electrical stimulation is similarly unselective, this selective integration process can similarly be used to drive stimulation of specific cell populations in tissue for other applications.

The integration between high density microelectrode arrays in the biohybrid implant and the host tissue also enables higher selectivity in the interaction with the target. In many applications it is desirable to not only record/stimulate a particular target tissue, but be able to selectively record/stimulate small portions of the target tissue selectively. By achieving a tight interface with many independently-controlled electrodes, the biohybrid medical device facilitates selective and independent stimulation/recording of portions of the integrated tissue. This degree of selectivity would be more difficult to achieve with devices which don't have this type of interface (due to larger distance between electrodes and tissue, and lack of a direct channel of communication between electrodes and tissue via potential biohyrid cell-tissue synapses) and/or have fewer/larger/less densely arranged microelectrodes.

The formation of a highly integrated tissue-implant interface, enabled by the cell type layer and other components of the biohybrid device, also offers another significant advantage in the field of implantable medical devices. Degradation of the implant-tissue interface is a major limitation to the operation of known medical devices for the long periods of time required, for example, for the useful delivery of treatments. Over time this interface tends to degrade due to a variety of factors, such as tissue damage and inflammation, fibrosis, foreign body reaction, implant material degradation, and implant failure. By forming a tightly integrated (both biologically and mechanically) interface with the tissue that is mediated by a biologically living (cell) layer, biohybrid devices avoid many of the processes driving this interface degradation, such as immune system recognition of the implanted materials at the interface and tissue damage due to micromovement.

Through the use of soft and flexible materials, biohybrid devices of the invention minimise other drivers of this interface degradation such as tissue-implant mechanical mismatch. Overall, the biohybrid devices offer advantages for the long-term stability and operation of implantable devices.

### Device Fabrication

The flexible electrode array can be built up from a substrate. For example, a substrate can be formed from a polymer (e.g. parylene C) layer that has been deposited on a silicon wafer (e.g. of 100 mm outer diameter, thickness of 1 mm), for example using an SCS Labcoater 2. The polymer layer can for example be 2 μm thick. Electrodes (preferably of gold) and interconnects can be patterned on the substrate, e.g. through a metal lift-off process. Photoresist can then be spin coated, baked, exposed and developed. For example AZnLOF9260 photoresist can be spin-coated at 3,500 r.p.m. on the substrate, baked at 110 °C for 120 s, exposed to ultraviolet light using a Karl Suss Contact Mask Aligner MA/BA6 and developed with AZ 760MIF developer.

A Ti adhesion layer (e.g. 10 nm-thick), followed by a Au layer (e.g. 100 nm-thick) can then be deposited (e.g. via an Angstrom EvoVac Multi-Process) and patterned (e.g. by soaking the substrate in a bath of acetone for 10 minutes).

A second (e.g. 2 μm-thick) layer of polymer (e.g. parylene C) can then be deposited to form an insulation layer, followed by spin coating a layer of soap solution (e.g. 2% Micro-90 diluted in deionized water) before an additional sacrificial (e.g. 2 μm-thick) layer of polymer (e.g. parylene C), for the subsequent peel-off process, is deposited. The layers of polymer are then patterned with another layer of positive photoresist (AZ9260) to shape the PEDOT:PSS electrodes and contact pads. This photoresist can then be dry etched, e.g. using reactive ion etching, to expose electrodes and contact pads.

Once etched, a thin film of PEDOT:PSS can be spin-coated onto electrodes. The solution can be spin coated several times, e.g. three times, with soft bakes in-between. The soft bakes may for example be for about 60 s at about 120 °C. After the final spin coat there can be a hard bake, e.g. for about 1 hr at about 130 °C.

Post-baking, the wafer can be left overnight in SI water to remove excess PEDOT:PSS. The following day the sacrificial layer of parylene C can be removed, leaving the finished flexible electrode array ready for use.

Although not illustrated or described, pattering of the electrodes and conductive lines could instead be performed via wet or dry etching of a metal layer. Another possible metal pattering technique is laser ablation of a conformal metal foil adhering to the base thin plastic layer.

### Device Preparation for Cell Culture

Custom-made polydimethylsiloxane (PDMS) wells can be attached to the flexible electrode array, for example using PDMS as a glue. The devices can be plasma treated, e.g. at about 25 W for about 1 min, to make the surface hydrophilic for cell culture. The inside of the well can be kept wet from this point on with DI water. The devices can then be entirely sterilized, for example for a minimum of 30 min in 70% ethanol and rinsed with Dulbecco's phosphate-buffered saline (DPBS).

### Cell Culture Preparation

The cells of interest, e.g. human iPSCs, can be expanded to give an adequate concentration. For example, human iPSCs can be defrosted and expanded in Essential 8™ Flex Medium for approximately 3 to 4 days in 6-well plates. This gives approximately 1.5 million cells/mL. The cells can then be seeded onto the flexible electrode arrays, for example with densities of 100,000 cell/cm².

Differentiation is preferably initiated 48 hours after cell seeding. MyoD media can be supplemented with fresh 1 μg/mL doxycycline (Sigma) and 1 μM Retinoic acid (Sigma) and 40 ng/ml of FGF2 (R&D). The cell culture media can be changed regularly, e.g. every day from day 0 to day 5. From day 6 onwards, MyoD media can be supplemented only with 1 μM Retinoic acid, 3 μM of CHIR99021 (Tocris) and 10% KOSR (ThermoFisher) and no doxycycline.

### Fibrin Hydrogel Preparation

Fibrinogen solution (SOL-FG) can be prepared from a fibrinogen stock, e.g. at a concentration of about 37.5 mg/ml in HEPES-buffered saline (HBS: 20 mM HEPES, 150 mM NaCl, pH=7.4) by slowly dissolving fibrinogen (F8630, Sigma Aldrich) for about 2 hours at about 37 °C (solution named SOL-FG). Calcium-Thrombin Solution (SOL-CaTh), e.g. containing 3 U/mL thrombin and 60 mM calcium ions can be prepared, e.g. by mixing equal volumes of SOL-Th and SOL-Ca, obtaining solution SOL-CaTh.

A solution containing 1,000,000 cells/mL in cell culture media (SOL-Cells) is then made.

This methodology is used to coat the cells that had been grown and differentiated on the polymer based bioelectronic devices.

For the production of fibrin gels, Equal volumes of SOL-FG, HBS and SOL-CaTh, with the cells SOL-Cells can be added on the desired vessels, preferably after incubating SOL-FG at around 37 °C for about 2 hours before mixing to allow gelation to occur. Once cells and fibrinogen are mixed, these solutions can be used within about 15 mins as cells/residual thrombin in cell culture media will start gelling.

Final concentrations: FG=3.125, 6.25, 12.5, 25 mg/mL, Th=1 U/mL, CaCl2=20 mM.

### Implantation Procedure

For implantation of the biohybrid device, an incision can be made, e.g. over the right forearm, between the shoulder and elbow of the subject. The muscle (e.g. the triceps muscle) can be lifted and the nerve bundle (e.g. the forearm combined ulnar and median nerve bundle) can be transected, e.g. at the height of the elbow.

The cell-laden side of a biohybrid implant device can then be positioned on the cut surface of the nerve and sutured, e.g. using a 9-0 nylon suture (Ethicon). The implant and nerve can then be moved as one unit, e.g. towards the midline of the subject and sutured to an anchor point, e.g. the latissimus dorsi muscle, for example using two 9-0 nylon sutures with the forearm nerve facing the muscle, and the back of the biohybrid device facing the skin of the forearm.

The dermis of the skin above the biohybrid device can be scored using a sterile blade, and the incision may then finally be sutured closed.

### Animal Study—Animal Preparation

190-240 g Hsd:RH-Foxn1rnu athymic nude rats (Envigo, France) were used in this study. Surgical procedures were performed under isoflurane anaesthesia, with the animal's body temperature regulated using a thermal blanket.

Implants were made according to the above methodology. Control implants (lacking cells) were implanted in an identical way. For implants later used in terminal recordings, biohybrid implants including a connecting FFC (flat flex cable) were used. The FFC is folded over once and tucked into a subcutaneous pocket running from the latissimus dorsi to the dorsal cervical region of the animal. Devices are otherwise implanted as above.

For implants later used in awake recordings, the FFC is run through a subcutaneous tunnel to the head of the animal. A custom-made 3D printed headcap is added as a port of access on the head and fixed using surgical screws and acrylic cement. A screw and stainless-steel wire are further implanted to connect to the CSF above the cerebellum and serve as ground during awake recordings. Animals are allowed to recover from the surgical procedure and provided with analgesics (Meloxicam, Carprofen) for two days post-implantation, as well as immediately prior to surgery.

Animals are housed in groups of three or four with ad libitum access to food and water.

### Animal Study—Electrophysiology Recordings Under Anaesthesia

28 days post-implantation, animals were anaesthetised using isoflurane. The FFC of their implanted device is exposed and connected to an electrophysiology acquisition and stimulation system (32-channel RHS headstage and RHS Stim/Recording Controller, Intan Technologies, USA). A pair of platinum wire hooks are implanted into the forearm nerve bundle-first of the contralateral, non-implanted forearm, and then of the operated forearm approximately four centimetres above the point of transection and implantation. The hooks are connected to the same acquisition and stimulation system. A ground shared by the recording MEA and stimulation hook electrodes is implanted into the subcutaneous space of the forearm. Hooks first implanted in the non-operated forearm nerve bundle are used to stimulate the nerve (square mono-polar pulse, 0.1 ms, 10, 50, 100 or 200 μA), and the amplitude at which a muscle twitch is induced is noted. Hooks are then moved to the implanted forearm nerve bundle. The stimulation paradigm is repeated, with 20-30 stimulation pulses delivered for each amplitude in each nerve, and the response is recorded through the biohybrid/control implanted device. Voltage signals are recorded and amplified (×192), bandpass filtered between 1 Hz and 7.5 kHz, and digitised at a 30 kHz sampling rate. Analysis of the pk-pk amplitude of the response to stimulation in the raw recorded signals is carried out in Spike2 (Cambridge Electronic Design, UK, v9.04b) using a custom script, and plots and statistical tests are carried out in Matlab (Mathworks, R2021b).

### Animal Study—Awake Freely Moving Rat Electrophysiology Recordings

Animals are positioned in a 0.3×0.3 m transparent arena. The headcap is opened and the FFC of the implanted device and ground are connected to a signal acquisition system. Animals were allowed to roam around the cage while video recordings were taken of them (GoPro Hero6 Black, GoPro). A green LED, driven by a digital-out port of the acquisition system, is used to align video and electrical recordings. Videos are manually annotated to indicate stepping events (reaching out and laying weight on the paw of the operated forelimb).

Recorded signals are visualised and quantified in Spike2 software (Cambridge Electronic Design, UK, v9.04b). Biohybrid device signals are produced by referencing pairs of electrodes within the MEA allocated randomly. These referenced signals are then bandpass-filtered (0.5-4 kHz, 4th order Butterworth filter). Signal-to-noise ratio (SNR) is calculated as the ratio of the variance during high signal relative to background activity, both identified manually, expressed as dB. RMS traces are produced from the referenced and bandpass-filtered signals by calculating the signals RMS (root-mean square) at 50 ms intervals and averaging the values using a 0.5 s rolling window.

Normalised signal is calculated by normalising each RMS trace (single recording session) to range from 0 (background noise) to 1 (highest amplitude signal). The fraction of signal amplitude occurring during step is calculated by comparing the average normalised RMS value during stepping events, relative to the same average value outside of these events. All plotting and statistical comparisons are carried out in Matlab (Mathworks, R2021b).

### Animal Study—Immunohistochemistry and Imaging

The whole tissue embedding and staining process occurred on a Leica Bond RX autostainer. All steps were performed within a vacuum at 40 degrees c for 1 hour. The steps were as follows: a wash in 70% Ethanol, 90% Ethanol, four 100% Ethanol washes, three xylene washes, followed by four liquid paraffin wax steps at 63 degrees C. The sections were first baked and de-waxed using Bond Dewax Solution (Leica Microsystems AR9222). Then the pre-treatment protocol was applied where Bond ER2 Solution is their pH9 antigen retrieval solution (Leica AR9640) at room temperature. The Bond Wash used throughout was AR9590. For the staining protocol a Bond Polymer Refine Detection kit (Leica DS9800) was used. The kit includes the peroxidase block, post-primary, HRP polymer secondary antibodies, DAB and haematoxylin.

The staining protocol began with 150 μL of peroxidase block added to the tissue samples and incubated for 5 minutes at room temperature. The sample was then washed with 150 μL of bond wash solution three times. Next 150 μL of the primary antibody, mouse monoclonal to human nucleoli [NM95] (Abcam ab 190710) for 60 minutes at room temperature. The sample is then washed with 150 μL of bond wash solution three times. 150 μL of the post-primary solution was incubated for 8 minutes at room temperature. Three 150 μL further bond washes were performed. Next, 150 μL of HRP polymer secondary antibodies was incubated for 8 minutes at room temperature. A 2 minute incubation with 150 μL bond wash solution was performed followed by a wash with 150 μL deionised water. Two washes with 150 μL DAB refine solution. 150 μL of Hematoxylin was added and incubated for 5 mins, followed by washes with 150 μL deionized water, 150 μL bond wash solution, 150 μL deionized water. Samples were then ready to be imaged.

Image analysis was performed in ImageJ software (National Institutes of Health). The edge of the tissue facing the device was traced by the user by hand and subsequently unfolded to become a flat image. Colour deconvolution was run to separate the implanted cells of interest (brown stain) from the host cells (blue) by difference in histology stain colour. The stain intensity values are then imported into Matlab (Mathwords, R2021b) to produce a mean intensity over distance from the implant using a custom script. Following this, a 400×400 pixel box was chosen in the original image in a region of tissue far away from the device and the average background stain intensity is measured. The intensity profile was divided by this value to produce a normalised intensity for each stain. Graph plotting and statistical comparison was carried out in Matlab.

### Animal Study—Methodology

The first step in the development of the biohybrid device was to choose an appropriate timeline to culture and sufficiently mature cells in vitro before implanting the device in vivo. Skeletal myocytes were selected and were generated from human induced pluripotent stem cells (iPSCs) by opti-ox cellular reprogramming as the biohybrid cell population. This system consistently produces highly pure myocytes after 8 days of culture. This made them well-suited to host sensorimotor nerves, whose motor axons typically innervate muscle tissue, while human iPSC-derived cells have the potential to provide off-the-shelf cell material for future clinical applications (clinically translatable). Based on the properties of the iPSCs-derived myocytes and of injured nerves, which typically regenerate within 3 weeks after injury, a timeline to test the ability of biohybrid devices to integrate with host nerves was determined.

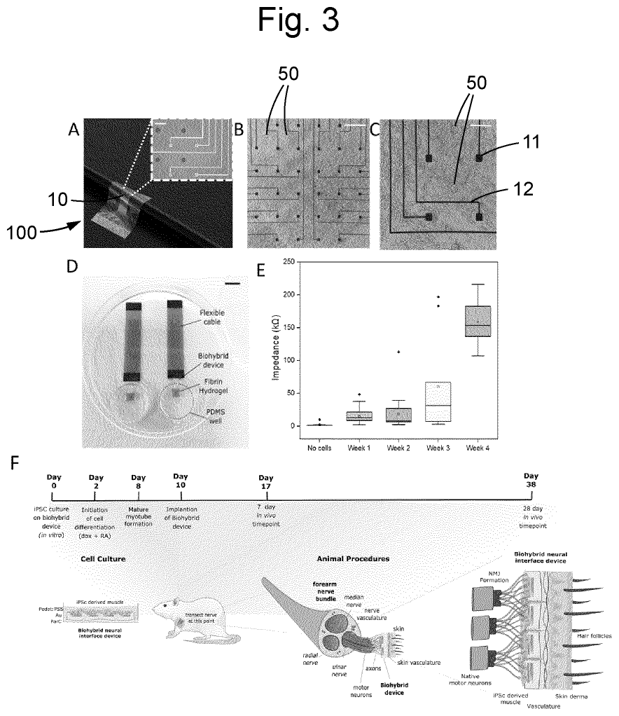

FIG. 3A shows an in vivo biohybrid device design customised for the peripheral nervous system. Scale bar: 60 μm.

The iPSCs were cultured on thin, flexible parylene-based microelectrode arrays (MEAs). The MEAs were fabricated using photolithography techniques to contain 32 conducting polymer (PEDOT:PSS) electrodes arranged in a symmetrical grid. The MEA occupied a 2×2 mm area within the larger parylene device, within which circular openings were introduced to permit growth of vasculature from the back of the device and support cell survival post-implantation. These openings 50 are visible in FIGS. 3B and 3C.

The culture process consisted of iPSC clusters being seeded on a fibrin hydrogel layer, to help encapsulate the cells on the device that is deposited on the MEA surface. The cultured cells can be seen in FIGS. 3B and 3C.

Following the induction of reprogramming 48 hours later, mature multinucleated myotubes are formed on the surface of the biohybrid device by Day 8, which (along with a study of acetylcholine (ACh) induced contractions) demonstrated myocyte maturity. The biohybrid devices showed good stability over the timeline used in the study, exhibiting an acceptable and expected increase in impedance over four weeks of in vivo culture—see FIG. 3E (prior to cell seeding: 97% yield, 1.84±2.20 kΩ; Week 4 in vivo: 25% yield, 159.00±35.80 kΩ, mean±SD).

FIG. 3F shows an experimental timeline showing the fabrication and implantation of the biohybrid device from an in vitro cell culture step into an animal model. Cells were seeded onto the flexible biohybrid devices at day zero. After 48 hrs (Day 2) the differentiation process is initiated. At Day 8 myotubes are mature, therefore between Day 8-10 is the optimal timing for the implantation of the biohybrid devices into a peripheral nerve rat model. Devices were then implanted for a period of 4 weeks.

The biohybrid devices containing mature myotubes have thus been implanted into immunodeficient rats. The immunodeficiency enhances human myotube survival, representative of systemic immunosuppression in humans—a strategy commonly used in cell transplantation studies. Biohybrid devices were implanted under the skin of the animal, with the MEA and cells facing the underlying musculature. The dermal layer onto which the device was laid was scored with a sterile knife immediately prior to device implantation to promote tissue regrowth and angiogenesis in the vicinity of the implant. This implantation strategy was seen to support cell survival for seven days after implantation.

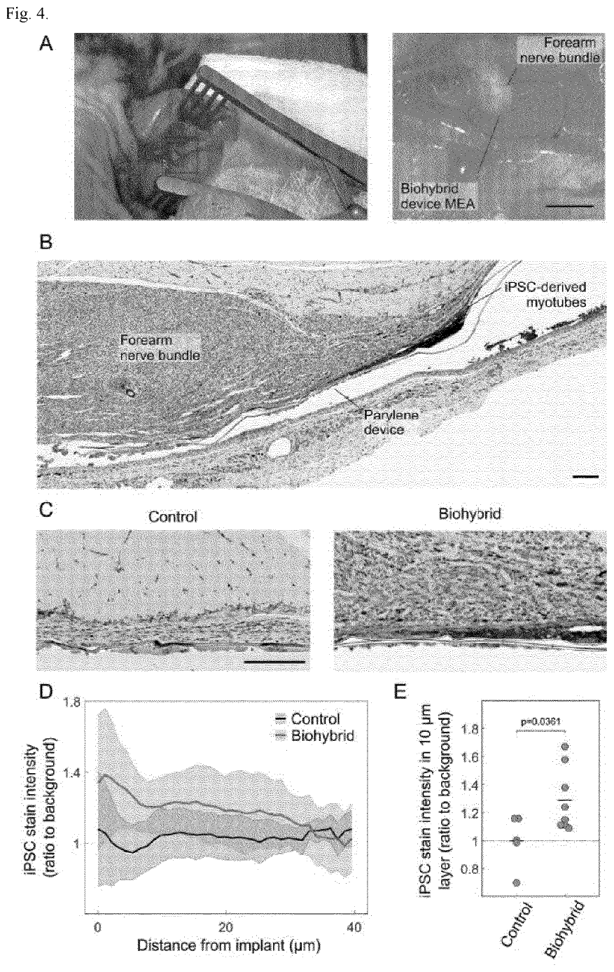

The ability of the biohybrid devices to host and integrate with a regenerating nerve was examined. The ulnar and median nerves were chosen, which control sensory and motor paw function and run together through the arm of the animal—hereafter referred together as forearm nerve bundle. This choice was driven by the clinical relevance of injury to upper limb nerves and higher degree of fine motor and sensory functions. Implantation was carried out by transecting the nerve bundle at elbow height and suturing the proximal nerve stump to the cell-laden side of the biohybrid device. The device was then transferred a few centimetres towards the midline of the animal and anchored subcutaneously above the latissimus dorsi muscle—see FIG. 4A-4B. Similar to the day 7 post-implantation observations, iPSC-derived cells were found to survive following four weeks of implantation with the sutured nerve—see FIGS. 4C-4E. The transplanted cells remained closely adhered to the biohybrid device following this period—see FIGS. 4D-4E. No major differences in host tissue appearance and cell density were seen across biohybrid and control devices—see FIGS. 4B-4C.

In FIG. 4E, average human nucleoli stain intensity in the 10 μm layer closest to the implant is shown. Biohybrid devices showed a significantly enriched presence of iPSCs compared to control devices. Circles in plot represent average in individual rats, black bar indicates mean of the group.

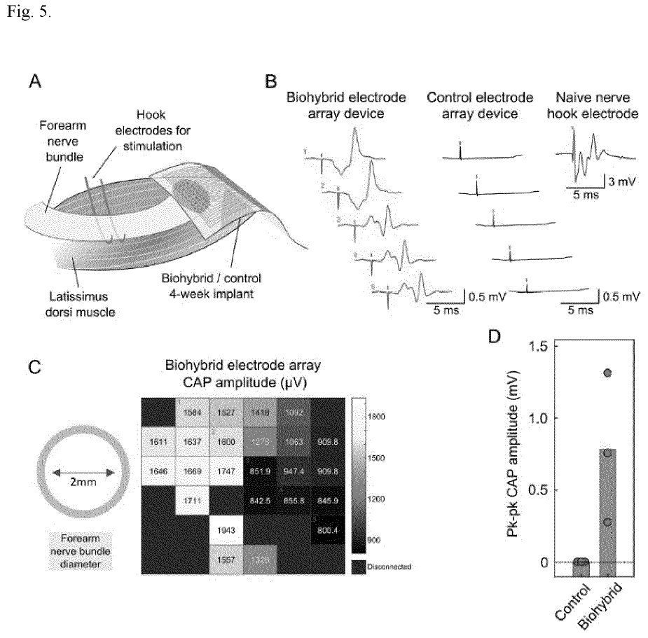

Good integration of the host forearm nerve bundle with the transplanted iPSC-derived myotube population was expected to result in improved nerve signal quality as recorded by the biohybrid implant. Nerve signals were recorded from nerves implanted with biohybrid devices and control devices lacking cells four weeks after implantation, a time-point by which nerve regeneration and integration into the nearby biohybrid cell population was expected to have occurred. At this timepoint, the connections of the implanted biohybrid/control device MEAs were exposed and a pair of hook electrodes were placed around the forearm nerve bundle approximately four centimetres above the point of transection. Using the hook electrodes the nerve was electrically stimulated and the response was recorded using the four-week implanted devices—see FIG. 5A.

FIG. 5A is a schematic of the experimental setup. 28 days post-implantation forearm nerve bundles that were transected and implanted with either biohybrid or control (lacking iPSCs) devices are exposed. Under anaesthesia, the nerve response to electrical stimulation is evaluated using the chronically-implanted devices.

The nerve was stimulated using a 0.1 ms duration pulse, for which a threshold of activation of 100 μA had been previously measured in the contralateral forearm nerve bundle. When the implanted nerve was electrically stimulated with a 100 μA pulse, a compound action potential (CAP) was recorded from biohybrid but not control implants—see FIG. 5B. This CAP consisted of a peak with an approximately 2 ms delay (corresponding to a conduction speed of ~20 m/s), consistent with Aα/β fibre activation, followed by a later peak, likely corresponding to reflex activity initiated by sensory fibre activation (H-reflex). These CAP features are consistent with those found in intact sensorimotor nerves—see FIG. 5B, indicating healthy nerve physiology in the transected forearm nerves implanted with a biohybrid device.

FIG. 5B, shows representative traces of nerve response to 100 μA, 0.1 ms stimulation pulses, taken from different electrodes within a device MEA. Compound action potentials (CAPs) are recorded from biohybrid (red) but not control (black) devices. CAP recorded from a naïve (non-transected) nerve using hook electrodes shown in blue for comparison. Stimulation time identified by cyan square in traces.

CAPs recorded over the entire biohybrid MEA significantly differed in amplitude. MEAs were designed with dimensions 2×2 mm, larger than the forearm nerve bundle (diameter of ~1 mm). This was designed to allow for the identification of different features in the recordings of electrodes under the nerve compared to those around it—see FIG. 4A. Hotspots in CAP amplitude in the MEA corresponded in dimensions to the forearm nerve diameter—see FIG. 5C, further supporting that the nerve had integrated with the biohybrid device over the implantation period.

FIG. 5C shows average pk-pk CAP amplitude from the MEA of a the biohybrid device from B. Higher CAP voltages represented by lighter colours in the heatmap, with exact values represented by the numbers. Electrodes with impedance >500 kΩ are considered disconnected and shown in black. The approximate dimensions of the forearm nerve bundle are indicated by the magenta ring. Individual electrodes from which the traces in FIG. 5B are taken are indicated by cyan numbers 1 through 5.

While all nerves implanted with biohybrid devices produced CAPs in response to 100 μA stimulation pulses, their average amplitudes differed, suggesting a degree of variability in biohybrid device integration across animals—see FIG. 5D.

FIG. 5D shows quantification of pk-pk CAP amplitude in 28 day-implanted biohybrid and control devices for 100 μA pulse stimulation. Control devices exhibit no CAPs in response to stimulation, while all biohybrid devices do. Red circles represent values for each animal (mean across entire MEA), bar indicates mean of whole group.

Consistent with normal nerve behaviour, stimulation with pulses of amplitude below activation threshold resulted in no CAP. Stimulation with much higher amplitude pulses eventually resulted in CAPs recorded in both groups. However, CAPs recorded by control devices under these stimulation conditions consisted exclusively of an H-reflex, which may have been mediated by reflex activity of other nerves at the same cervical level as the forearm nerve bundle.

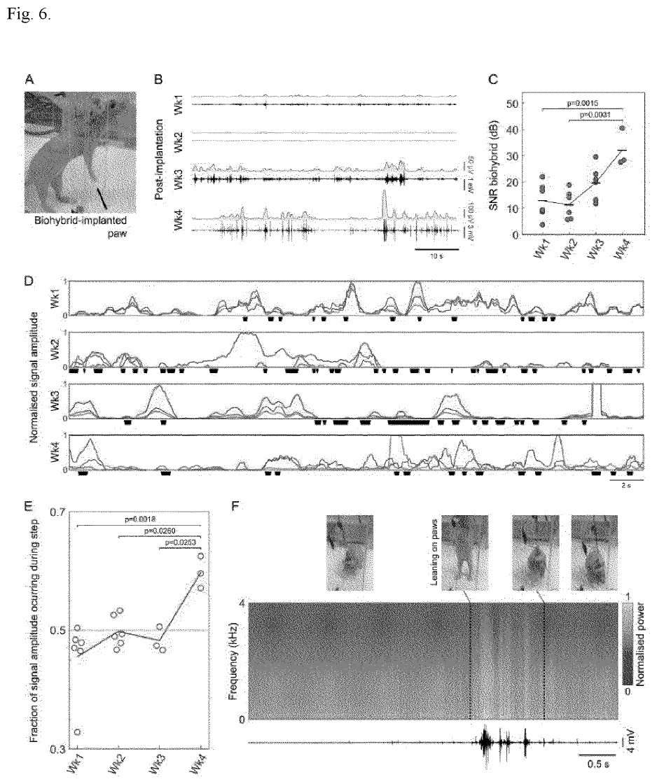

In order to track the integration of nerve with biohybrid device, two animals with biohybrid devices were implanted in a similar fashion and the MEA connections were externalised through a headcap. Over the course of four weeks, weekly recordings were performed through the implanted biohybrid device while the animal roamed freely in an arena—see FIG. 6A. FIG. 6A is a photograph of the experimental setup. Rats are implanted with a biohybrid device into their right forearm nerve bundle, with the connections externalised through a headcap. This permits awake recordings over weeks post-implantation.

To minimise EMG noise from nearby musculature, bipolar recording electrodes were set up between pairs of electrodes across the biohybrid MEA. These pairs were maintained consistent throughout the four weeks of implantation. Within the first two weeks of implantation little activity in the awake animals was observed. By the third and in particular the fourth week, however, signals had greatly and significantly increased in amplitude—see FIGS. 6B and 6C.

FIG. 6B is a forearm nerve signal traces recorded from biohybrid devices over four weeks post-implantation. Black: bandpass filtered (0.2-4 kHz) time traces recorded from a pair of electrodes in the MEA (bipolar configuration). Cyan: root-mean square (RMS) of black traces with 0.5 s rolling window. Nerve signal amplitude increases over implantation period.

FIG. 6C is a quantification of signal-to-noise ratio (SNR) of recorded traces. Red circles: mean value per bipolar electrode, black line: mean of group, N=2 rats. Statistical comparison done via one-way ANOVA followed by Tukey post-hoc. P values not shown are >0.05.

As axon regrowth usually begins 4 days after injury, and accounting for the small distance between nerve and biohybrid device, the recorded increase in signal amplitude matched the expected timeline of axon regrowth into the biohybrid device. The integration of the forearm nerve bundle into the biohybrid device was expected to result in activity which correlated with paw function, normally driven by this nerve. The correlation of electrical activity recorded by the device with paw function while the animal roamed around the cage was then evaluated. The forearm nerve bundle controls paw function. However, as it had been transected as part of the implantation procedure paw or digit movement could not be used as an indicator of nerve activity. Instead, stepping movements were identified, which would normally end with the extension of the digits as the paw approaches a surface, and these events were matched to recorded electrical activity—see FIG. 6D.

FIG. 6D is RMS time traces from three bipolar electrodes (cyan, green, magenta) over four weeks of implantation, normalised to range from 0 to 1 for each week. Points when the animal was reaching out and stepping with the implanted paw is indicated by black squares under each trace. Increased correlation between recorded activity and paw movement, indicative of good forearm nerve recording, develops at week 4 post-implantation, with less activity seen outside of stepping events.

At early implantation timepoints it was observed that recorded activity appeared to be independent of movement of the operated limb. However, by week four post-implantation activity increasingly correlated with movement of the implanted paw—see FIGS. 6D-6F.

FIG. 6E is quantification of correlation between recorded activity and stepping events with implanted paw. Cyan circles: mean fraction of RMS activity occurring during steps. These values are adjusted to account for time stepping/not stepping in that session. Red line: mean of group, N=2 rats. Statistical comparison done via one-way ANOVA followed by Tukey post-hoc. P values not shown are >0.05.

FIG. 6F is a sample time-frequency spectrogram and time trace of a recording from a bipolar electrode at week 4 post-implantation. High activity is seen when rat steps up and leans on the behaviour chamber wall with paws (splaying of digits visible in non-implanted forearm, which retains an intact forearm nerve bundle, delimited by black dotted lines). Comparatively less activity is seen before and after this event, as animal remains on the bottom of the chamber, indicating that recorded activity is driven by attempted paw use.

We also observed that by week four different electrode pairs often recorded activity independently of each other—see FIG. 6D. This may indicate that different axon bundles of the forearm nerve bundle, which typically innervate different muscle groups around the paw, had grown into different regions of the MEA. This is further supported by the large increase in activity amplitude which developed at this timepoint—see FIGS. 6B-6C. Activity at earlier timepoints was instead typically recorded across all electrodes. Other changes, such as a moderate increase in electrode impedance, were also observed between week four and previous timepoints—see FIG. 3E. These observations support that following four weeks of implantation the host nerve had grown into and integrated with the biohybrid device, greatly improving recording performance.

The biohybrid medical device of the invention has particular application to operate a prosthetic (e.g. a prosthetic limb) through, for example, implantation into nerves of the forearm (radial and median nerves) in hand amputee patients at the upper arm level. Integration of the median and radial nerves into the biohybrid device would enable the recording of high-quality electrical signals corresponding with motor signals carried by these nerves. In non-amputees these motor signals directly control the contraction of muscles in the forearm and hand, which cause movement of the hand. In amputees implanted with this biohybrid systems, the recorded signals can be used to guide an active prosthetic hand.

Movement of the prosthetic hand is carried out by first decoding the contraction of which specific muscle corresponds to different recorded nerve activity. Different anatomical portions of the nerve (fascicles) innervate different portions. The high spatial resolution and selectivity enabled by the biohybrid device is thus of advantage to carry out this decoding.

Once correlated with distinct muscle contractions, a hand prosthetic capable of movements similar to those of the human hand, powered by micromotors, can be driven by the recorded activity. This can be carried out by extracting an amplitude-over-time metric from the recorded activity. When a minimum threshold of amplitude is reached, the prosthetic motors performing the movement corresponding with the high amplitude recording can be activated. The movement can be maintained while recorded amplitude remains above a given threshold, and deactivated when amplitude drops below this threshold.

In this example, overall this would allow the driving of a motorised hand prosthetic via recording of motor signals from median and radial nerves.

The invention has been validated with the above example focused on skeletal myocytes applied to motor nerves, for example for application in prosthetics. However, biohybrid medical devices can be implemented in other configurations, for alternative applications and using alternative manufacturing methods.

Different biological cells aside from skeletal myocytes can be used to form the interface between the biohybrid device and the tissue. Neurons can be used to either receive or provide synaptic connexions to other neurons (by growing axons out of the biohybrid device and into tissue or permitting the growth of axons from tissue into the biohybrid device). This can be applied in locations such as the brain, spinal cord, and peripheral nerves. Given the characteristics of the cells used and locations suitable for implantation, such a configuration of a biohybrid device can be used to neuromodulate specific locations and neuronal types in the target tissue, for applications equivalent to deep brain stimulation, spinal cord stimulation for neuropathic pain management or movement and/or sensation restoration, cortical stimulation for visual restoration, brainstem stimulation for auditory restoration, among others. The device can also be used to selectively record neuronal activity from certain neuronal populations (specific locations and neuronal types), in applications such as the driving of prostheses via cortical brain activity in a brain-computer interface setup.

The use of fibroblasts as the interface cells allows all the benefits of the biohybrid in reduction of foreign body reaction and tissue integration and can be adoperated in to enhance electrically-driven wound healing and tissue repair.

In addition to, or as an alternative to, thin-film parylene and PEDOT:PSS electrodes, biohybrid devices may be fabricated using different techniques and materials. Devices manufactured using other materials can achieve similar low bending stiffnesses to parylene C, such as PDMS and polyimide. These materials can offer advantages for points critical to biohybrid function such as successful culture of a stable cell layer on the device. This includes chemical surface modification, and loading with chemoattractant or cell modulating chemicals for slow release. Similarly, different conductive polymers may offer other advantages as electrodes over PEDOT:PSS, such as higher stability for electrical stimulation and lower stiffness for higher biocompatibility. In addition to microfabrication in the described methodology of the devices, other fabrication methods may offer other advantages. Screen printing, for example, may be used to increase device fabrication output and lower fabrication costs when high-resolution of device features are not required.

There is thus disclosed herein a new category of neural interface, which combines flexible electronics and cells, preferably human iPSC derived cells. The implanted human iPSC derived myocytes can survive in a rat model for up to 28 days and integrate with the host tissue and then be used to restore and drive functionality through a biohybrid device. The biohybrid neural interfaces of the invention show superior electrophysiology recording properties compared to standard neural interfaces (e.g. electronics without cells). The invention opens the door to the development of treatment of multiple neurological diseases, such as stroke, brain, spinal and peripheral nerve injuries. By selecting the appropriate implant design and cell type, customisable biohybrid neural interfaces can be generated to meet patients' individual requirements. Furthermore, the combination of an implanted device and cells allows, through bespoke genetic modifications of parental iPSC for instance, for the use of local drug delivery, for immunosuppression or growth factor delivery.

### Use of Cell Components

While the foregoing description has referred to medical devices comprising a culture of entire biological cells, the invention is not limited thereto. Rather, medical devices of the invention may comprise, instead of a culture of entire biological cells, a plurality of biological cell components on the flexible electrode array. Such medical devices, comprising cell components instead of entire cells, may exhibit many of the same advantages as the medical devices referred to in the foregoing description. Herein, “biological cell component” refers to a part of a biological cell in isolation. As is understood in the art, there are various components that form biological cells, such as cell membranes, nuclei, ribosomes, etc. A medical device of the present invention may therefore be substantially as described in the foregoing description but comprising, as opposed to a culture of entire biological cells, a plurality of cell membranes (separated from the other components of the cells from which the membranes are derived) deposited on the flexible electrode array. Preferable cell components for the present invention are neuronal membranes, with a particularly preferable example being human iPSC-derived neuronal membranes.

Accordingly, any of the medical devices described herein as comprising a culture of biological cells may comprise alternatively to living cells a plurality of cell components. Likewise, any of the methods of making medical devices described herein may comprise alternatively to a step of culturing living biological cells, a step of depositing biological cell components on the flexible electrode array. Likewise, any of the methods of implanting medical devices in a subject described herein may comprise suturing a cell component-laden side of the device to tissue in the subject. Likewise, any of the methods of treating a human or animal body described herein may comprise implanting a medical device which comprises cell components rather than living cells.

In an embodiment, human iPSC-derived neuronal membranes are used as a biological mediator between the device and host tissue. This addresses the foreign body reaction (FBR), resulting in reduced inflammation and improved stability. As discussed further below, it has been found that neuronal membrane-derived biohybrid neural interfaces (NMemb-Biohybrids) implanted subdurally in rats result in reduced inflammation post-implantation at day 28 compared to those without, while maintaining high signal-to-noise ratio of electrophysiological recordings in vivo. These findings hold great promise for enhancing biocompatible neural interfaces and chronic therapeutic interventions, and suggest the possibility of improving device performance and integration through exploiting and controlling lipid and protein composition at the nanometer scale.

As described herein, incorporating a physiological barrier between the electronic device and brain tissue can help mitigate FBR, extend device longevity, and lessen adverse effects on native brain cells. The use of cell components as a barrier rather than living cells is advantageous due to the fact that survival of cells introduces additional complexity to clinical translation, particularly in uninjured tissues. The use of cell components such as cell membranes also reduces an immune response to the biohybrid implant.

In the present embodiment, the biomimetic component of the biohybrid neural interfaces are mostly related to the presence of the cellular membranes. These are specialized barriers made of phospholipids and proteins that regulate interactions between cells and their external environment, with transmembrane proteins play a fundamental role in a variety of cellular processes, including transport and signaling. Promising advancements have been made to monitor cellular membranes, including the use of planar lipid bilayers, referred to as supported lipid bilayers (SLBs), formed on cushioned surfaces and bioelectronics. The formation of SLBs through the extraction of blebs from cultured cells (referred to as “native SLBs”) allows for the preservation of the native environment's compositional complexity, including transmembrane proteins, as well as eliminating the need for laborious lipo-protein reconstitution experiments. Leveraging the potential of these native SLBs as a biological mediator, they can be deposited onto flexible electronics, thereby serving as an intricate intermediary between the electronics and host tissue in vivo to mitigate the inflammatory response, although the insulating nature of these lipids may compromise the device's recordings.

In the present embodiment, the modularity of cells is harnessed to mitigate the complex biological mechanisms involved in FBR through an in vivo neuronal membrane-derived bioelectronics model. Using bioengineering approaches and well-established flexible electronics (based on poly(3,4-ethylenedioxythiophene) polystyrene sulfonate (PEDOT:PSS) electrodes), a neuronal membrane biohybrid bioelectronics (NMemb-Biohybrids) is designed based on human induced pluripotent stem cell (iPSC)-derived glutamatergic neurons to form biologically complex SLBs (neuronal membranes) on flexible bioelectronics for subdural implantation. This approach on a rat brain model has shown to be successful in significantly reducing the FBR capsule while maintaining high signal-to-noise ratio (SNR) recordings in vivo using electrocorticography arrays (ECoG). This opens the door for the next-generation of biocompatible neural interfaces by exploiting cellularly-driven modularity. By manipulating the design at the nanometer level, this demonstrates the ability to further enhance the performance, integration and longevity of these devices, allowing for more precise and reliable measurements of neural activity while integrating seamlessly with our biology aimed towards chronic interventions.

Fabrication and experimental results in relation to the present embodiment are discussed below.

Characterization and Validation of Neuronal-Derived SLBs Integrated with Bioelectronics in Vitro

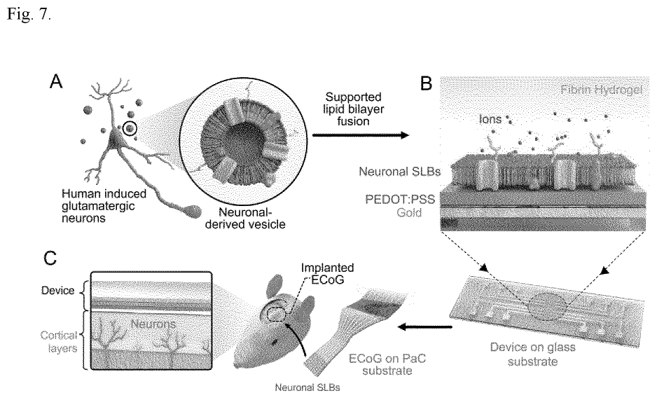

The technique for isolating lipid vesicles from plasma membranes of iPSC-derived glutamatergic neurons is a relatively novel approach (FIG. 7) and it is essential to validate and characterize the fused membranes to examine their optical mobility and electrical sealing properties in vitro (FIG. 7A-B). First, the cells were selected as they display an accelerated maturation process by day 8, resulting in fully functional and clinically translatable glutamatergic neurons. These cells were developed using OPTi-OX reprogramming technology, which is predominantly characterized by the expression of the VGLUT1 and VGLUT2 genes and additional classical cortical marker genes such as FOXG1 and TBR1. Upon their revival in the appropriate media supplemented with doxycycline, the membranes were isolated and fused on bioelectronic devices. The neuronal-derived SLBs were then encapsulated with fibrin hydrogels prior to implantation, retaining their hydration (FIG. 7B—fibrin hydrogel). NMemb-Biohybrids were implanted in rat models for 28 days to test and analyze the in vivo model for mitigating FBR (FIG. 7C).

In more detail, FIG. 7 shows blebbing of human iPSC-derived glutamatergic neurons at day 8 of cell-culturing (FIG. 7A) and the subsequent fusion of the neuronal-derived vesicles into SLBs (using fusogenic liposomes composed of a DOPC/DOTAP mixture and PEG) encapsulated within fibrin hydrogels on bioelectronic devices (FIG. 7B). To evaluate these biologically complex SLBs, an in vitro 12-electrode microelectrode array (MEA) was used. The device consisted of Au electrodes and a PEDOT:PSS cushion (200 μm×200 μm) on each electrode, with native membranes deposited on top (FIG. 7B; bottom device). An in vivo bioelectronic device (using flexible ECoG arrays on Parylene C (PaC) substrates) design customized for subdural implantation into animal models (FIG. 7C).

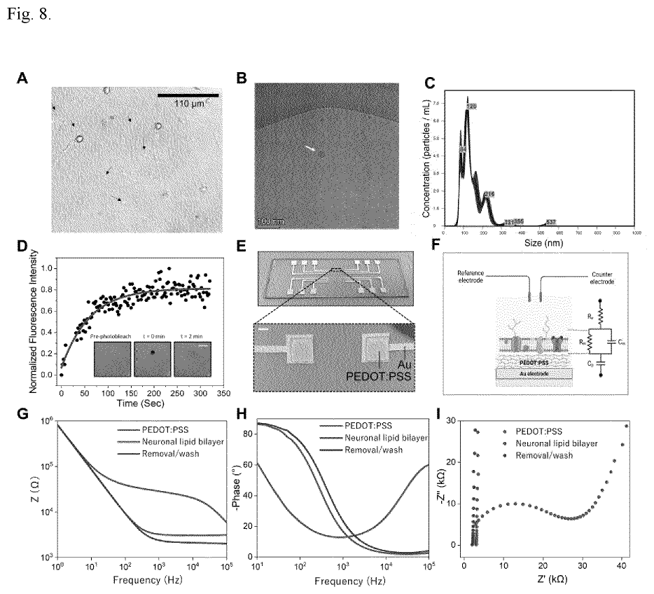

By day 8 of cell-culture, the blebbing process was chemically induced where the plasma membrane is budding and vesiculating. FIG. 8A shows blebbing of iPSC-derived glutamatergic neurons at day 8; cultured neurons undergoing blebbing under a 20× magnified, grayscale brightfield image, demonstrating the budding and vesiculation of their membranes after 2.5 hour incubation in the vesiculation buffer. Note: An area of sparse neurons was intentionally selected to clearly demonstrate the vesiculation of the cell membranes. After cellular blebbing, the collected lipid vesicles display size heterogeneity, ranging from 84-216 nm in diameter, with some measuring up to approximately 500 nm, and per transmission electron microscopy (TEM) micrographs (FIG. 8B), they exhibit spherical structures, as desired (FIG. 8C shows a polydisperse solution of collected lipid vesicles measured using an NTA, showing a concentration vs. size plot, with the highest concentration at 120 nm). This heterogeneity is likely due to the presence of smaller nano-meter-sized blebs as well as giant plasma membrane vesicles (GPMVs), which likely encompass a higher density of proteins than their smaller counterparts. These neuronal vesicles have particle zeta potentials of −13.9±0.9 mV. The mobility and lateral diffusion of lipid bilayers are defining features of their native environment. Developing a biomimetic model that replicates this behavior requires neuronal-derived membranes to exhibit similar characteristics. To create and fuse these SLBs, a low concentration mixture of synthetic liposomes and polyethylene glycol (PEG) was utilized to induce the rupture of vesicles on solid surfaces, acting as nucleation sites. By employing smaller synthetic vesicles with high curvature (<90 nm), they are more likely to rupture, assisting in the rupture of larger vesicles, and leading to their subsequent fusion.

To confirm the fusion of the neuronal-derived SLBs, fluorescence recovery after photobleaching (FRAP) experiments were conducted. A high intensity laser photobleached an area of the fluorescently labelled SLBs (using 0.36 mM of R18 dye) once formed on PEDOT:PSS films where the fluorescence intensity recovery is continuously monitored (FIG. 8D, showing FRAP measurements after deposition of fluorescently labelled neuronal-derived lipid vesicles (0.36 mM R18 dye) on PEDOT:PSS films after their adsorption, rupture and subsequent fusion via self-assembly with the aid of fusogenic liposomes (DOPC/DOTAP mixture, DOPC: 1,2-Dioleoyl-sn-glycero-3-phosphocholine, DOTAP: Dioleoyl-3-trimethylammonium propane) and PEG). As seen, these SLBs are highly mobile on PEDOT:PSS, demonstrating a high mobile fraction (MF) and diffusion coefficient (D) comparable to those of glass surfaces, serving as a control substrate (D=2.87±1.06 μm²/s, MF=0.87±0.14 and D=4.34±1.54 μm²/s, MF=0.82±0.07, respectively).

To quantitively measure the barrier-forming SLBs, EIS spectra can be used to extrapolate membrane resistance values by fitting the Nyquist plot and modelling of the electric equivalent circuit (EEC). The SLB-PEDOT:PSS-Au devices' electric circuit can be illustrated as follows, which aligns with prior studies using Au/PEDOT:PSS microelectrodes (FIG. 8E-F). A parallel connection of membrane resistance (Rm) and capacitance (Cm) is linked to a series connection of electrolyte resistance (Re) and PEDOT:PSS capacitance (Cp). Using regression analysis, the state of the SLB at the time of measurement can be quantified and membrane properties (i.e., sealing properties) can be inferred using EIS. In particular, FIG. 8E shows Au/PEDOT:PSS microelectrodes consisting of varying geometries, including 200 μm×200 μm electrodes, which was used as the most suitable dimensions to sense these membranes (scale bar=100 μm) and FIG. 8F shows EEC modelling of the membrane used to extract membrane resistance from EIS spectra.

FIGS. 8G-I show Bode and Nyquist plots of deposited neuronal membranes. The device underwent measurements in PBS using Au/PEDOT:PSS electrodes, as indicated by the blue line. Subsequently, the neuronal membranes were fused on Au/PEDOT:PSS electrodes, and measurements were taken resulting in the purple line. Afterward, the membranes were washed and removed and measurements were taken again as illustrated by the black line. The impedance vs. frequency (FIG. 8G) and phase shift vs. frequency (FIG. 8H) of the Au/PEDOT:PSS response to membrane depositions are shown. The Nyquist plot (FIG. 8I) displays the shift to higher impedance values upon SLB addition, with complex imaginary (Z″) versus real (Z′) impedances being plotted (n=5). The electrode size of 200 μm×200 μm showed the greatest increase in impedance upon SLB deposition, indicating a highly sealed bilayer (blocking ionic flow into PEDOT:PSS) with a calculated membrane resistance of 14.4±6.8 kΩ, the highest of all dimensions explored (FIG. 8G). The impedance values of Au/PEDOT:PSS microelectrodes (SLB-free) were measured first followed by the deposition and fusion of the neuronal membranes with Z=3.7×10⁴Ω at 1000 Hz (an increase of 15-fold compared to bare PEDOT:PSS electrodes with 2.4×10³Ω at 1000 Hz; FIG. 8G). Upon removing the SLBs, the impedance values recover back to their original values, indicating that these electrochemical changes observed were due to the presence of the robust (low-leaking) membranes. This trend is also visible in the negative phase changes once the SLB is added by the additional time-constant and shift to higher impedance values in the Nyquist plot (FIG. 8H-I).

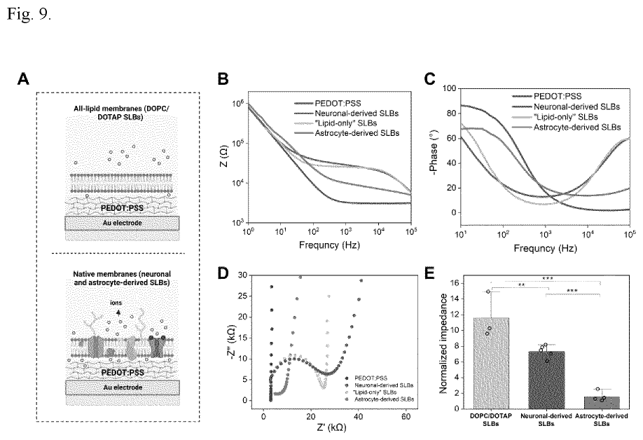

To test the novel approach, the electrical properties of human iPSC-derived neuronal membranes were investigated in comparison to a control group consisting of non-neuronal native membranes, specifically astrocyte membranes. Native membranes, in this context, refer to membranes obtained directly from cellular blebs. Astrocytes were used to create the native membranes by inducing their rupture on PEDOT:PSS-based surfaces using fusogenic liposomes and PEG. Then the quantitative electrical data extrapolated via EIS spectra of these native membranes were compared to lipid-only bilayers (made of a mixture of only DOPC/DOTAP) and the membranes created for iPSC-derived neurons. The diverse protein composition of native membranes likely leads to “leaky” membranes (or defective areas) with increased permeability to ions and lower impedance (at 10³ to 10⁵ Hz) compared to lipid-only membranes, which have significantly higher impedance and better sealing properties due to their tight packing of lipids and lack of pore-forming proteins. This was observed as seen in FIG. 9.

In comparing different types of self-assembled lipid bilayers on electrodes, their electrochemical properties demonstrate distinct behaviours (FIG. 9A). FIG. 9A (created using Biorender.com) shows a schematic demonstrating the increasing complexity of SLBs: “Lipid-only” membranes assembled via vesicle fusion of unilamellar synthetic vesicles vs. complex native membranes that are derived from cellular blebs (simplified image). It displays the varying sealing properties of these SLBs once self-assembled on Au/PEDOT:PSS microelectrodes with dimensions of 200 μm×200 μm.

FIGS. 9B-D show: Bode plots of bare electrodes (PEDOT:PSS; black line), neuronal (purple line) and astrocyte-derived SLBs (pink line) and DOPC/DOTAP SLBs (green line), showing impedance (FIG. 9B) and phase shift (FIG. 9C) as a function of frequency as well as the Nyquist plot (FIG. 9D). FIG. 9E shows normalized impedance for each SLB type. As shown, the “lipid-only” SLBs, composed of a DOPC/DOTAP mixture, show a dominant resistive component, resulting in an initial plateau across mid-high frequency in the Bode plot (FIG. 9B-C; green). In contrast, the “PEDOT:PSS-only” devices exhibit a clear declining slope at low-mid frequency, indicating a capacitive component (FIG. 9B-C; grey). Adding astrocyte-derived membranes to the electrodes results in SLBs that are less resistive than “lipid-only” membranes but still display capacitive and resistive properties (FIG. 9B-C; purple). The neuronal SLBs developed exhibit a high-quality barrier, dominated by the resistive component similar to the DOPC/DOTAP SLBs (purple). The astrocyte-derived SLBs show a low-quality barrier (or lack thereof), compared to the neuronal and “lipid-only” ones. The membrane resistance values follow the trend of “lipid-only”>neuronal>astrocyte-derived SLBs (Table 1, below).

TABLE 1
A comparison of Rm of “lipid-only” vs. biologically
complex SLBs on Au/PEDOT:PSS microelectrodes.

| | DOPC/DOTAP | Astrocyte-derived | iPSC-derived |
|---|---|---|---|
| SLB type | SLB | SLB | neuronal SLB |
| Rₘ (kΩ) | 24 ± 1.0 | 0.8 ± 0.5 | 6.7 ± 4.2 |

n = 3-5 for each group (average ± s.d.). Rₘ = membrane resistance.

The results of this investigation indicate a significant increase in fold change between “lipid-only” bilayers and astrocyte-derived SLBs (p=0.000033; n=3-4), as well as between neuronal SLBs and astrocyte-derived SLBs (p=0.00084; n=4-5). The “lipid-only” bilayers are also significantly higher than neuronal SLBs (p=0.01; n=3-5). These findings provide further evidence that the developed neuronal SLBs maintain a high-quality barrier on Au/PEDOT:PSS-based microelectrodes, akin to the robust barrier observed with DOPC/DOTAP SLBs in FIG. 9D-E. However, the presence of pore-forming proteins in neuronal SLBs contributes to a certain degree of leakiness, which is a desirable feature in the NMemb-Biohybrids. Variability in resistivity is attributed to SLB composition, degree of heterogeneity, and experimental differences in blebbing procedures.

### Investigating Inflammatory Response to NM-Biohybrid In Vivo: Focus on Activated Microglia and Reactive Astrocytes

Due to their instability in dry conditions and the challenge of processing lipid-based materials for histological analysis, the use and application of lipid bilayers in vivo are limited. Therefore, identifying ways to enhance neuronal membrane stability and detecting them in regions of interest post-procedure or implantation is crucial. To achieve this, the neuronal membranes were encapsulated with a highly porous, ion-permitting hydrogel within the bioelectronic device. Fibrin hydrogels are a natural polymer and have been widely used in medical devices due to their degradability and biological origin to improve biocompatibility/device-tissue integration. In vitro, the fibrin hydrogel encapsulating the neuronal-derived SLBs showed no significant difference in mobility using FRAP as well as electrical sealing properties via EIS spectra.

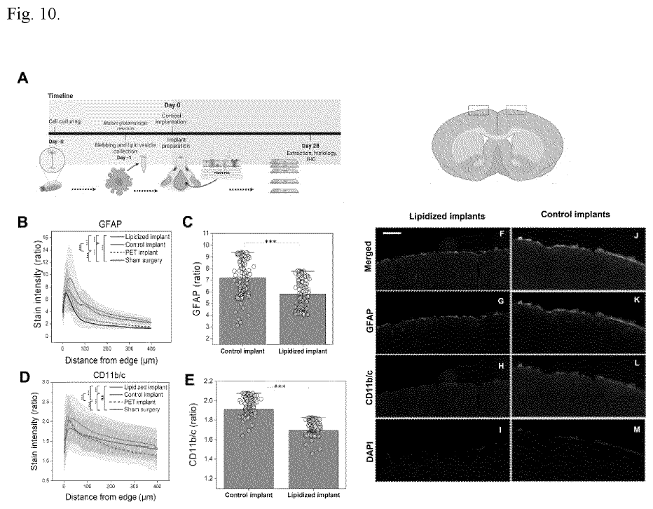

Aspects of the study which is discussed below are illustrated in FIG. 10. FIG. 10A (created using Biorender.com) shows: a timeline of cell culturing iPSC-derived glutamatergic neurons for 8 days, the cells underwent blebbing and vesicles were collected to assemble the neuronal membranes on implants encapsulated with fibrin hydrogel. At day 0, lipidized implants (PEDOT:PSS-coated) and control groups were subdurally implanted for 28 days. Brains were then extracted for histology and immunohistochemical analysis of GFAP and CD11b/c to assess FBR to neural implants. Immunohistochemical analysis of FBR capsule (GFAP) after implanting the NMemb-Biohybrids at day 28 with stain intensity profiles of GFAP (FIG. 10B) at 400 μm thickness from the edge of the cortex in which the implant is interfaced, analyzing four groups: lipidized implants, control implants (−SLB), PET implants (positive control) and sham surgery group. All group combinations of GFAP intensity are significantly different (p<0.001), using a two-way ANOVA and post-hoc tests. Averages of the stain intensity of GFAP of the first 100 μm layer closest to the implant (FIG. 10C). Lipidized implants are significantly lower than control implants in (p=4.4×10⁻⁷) using a one-way ANOVA and post-hoc tests. Stain intensity profiles of CD11b/c (FIG. 10D) at 400 μm thickness from the edge of the cortex at the site of implantation. All group combinations are significantly different (p<0.001), except when comparing sham surgery group to lipidized implants (p=0.389). Averages of the stain intensity of CD11b/c of the first 100 μm layer closest to the implant (FIG. 10E). Lipidized implants are significantly lower than control implant in CD11b/c (p=9.5×10⁻²⁰). Immunofluorescent images after extracting the brains at day 28, cryosectioning, and staining with GFAP, CD11b/c, and DAPI to observe the effects of FBR and inflammation degree to the biohybrid model at the implant site. Animals received lipidized implant (FIG. 10F-I) and control implant (FIG. 10J-M). These images represent a single animal. Scale bar=250 μm. Mean (solid line)+s.d. (shaded area). N=11 animals, ***p<0.001.

The study focused on detecting lipid membranes implanted in the brain after 1, 7, and 28 days, using fluorescent labeling with a lipophilic tracer (0.25% SP-DiLC18). Stacking multiple lipid layers was done to maximize detection. Results showed high fluorescence intensity on day 1 (normalized fluorescence intensity=723±459 a.u.) (mean±s.d.), indicating survival of the implanted lipid membranes, but a significant decrease in intensity by day 7 (112.3±112.6 a.u.) likely due to the immune response to the implant. Fluorescence intensity was even lower on day 28 (34±32 a.u.), indicating resolution of the foreign body response by phagocytosis. While drawbacks to fluorescence labeling were noted, such as seeping of dye into tissue and weakening of signal over time, which potentially is due to the shedding of lipid layers or loss of membrane during extraction, the results support the presence of the neuronal membranes at the implant-tissue interface at early timepoints after implantation. Corrected total fluorescence at the region of interest was calculated using integrated density and background fluorescence.

This study included four groups: sham surgery, PET implant (positive control), control implants with fibrin hydrogel (SLB-free), and lipidized implants with neuronal membranes and the hydrogel. PET implants were chosen as positive controls as we expect a stiff material to yield a higher FBR. The immune response was then investigated using immunohistochemistry to assess the FBR severity across the model. The analysis involved plotting stain intensity profiles and using Matlab code to scan the FBR capsule image pixel-by-pixel. GFAP (a biomarker for astroglial injury) and CD11b/c (microglia/macrophage biomarker) stains were used to differentiate the four groups, and the study involved cell culture for 8 days prior and cortical implantation at day 0, with brain extraction at day 28, followed by immunohistochemical staining afterwards (FIG. 10A). The timepoint was chosen to investigate the initial stages of acute FBR and to determine whether FBR worsened or resolved in response to the implants.

The first sign of FBR capsule formation in the brain is the presence of reactive astrocytes, activated microglia, monocytes, and macrophages. After imaging and staining the brain sections, GFAP stains were measured and further analyzed as part of the FBR capsule created around the implant on day 28 (FIG. 10B-C). The PET implant exhibited the highest intensity peak, followed by the control implant without neuronal membranes, and finally the implants with neuronal membranes (FIG. 10B). The stiffness of PET along with the thickness of the implant (nearly 100× thicker than PaC implants) might cause negative effects in the tissue. In comparing the control implant without neuronal membranes and the lipidized implant at the region of interest, covering nearly 400 μm distance away from the implant, the NMemb-Biohybrids showed a significantly lower GFAP stain profile with a quantified peak stain intensity (at 23 μm) at 7.5±3.4 (p=4.8×10⁻¹²), compared to the control implant (peak stain intensity at 31 μm with a ratio of 9.35±5.6) and PET (positive control) with peak stain intensity at 9.8±2.2 (18 μm) (FIG. 10B). Among the tested implants, those with lipidized surfaces exhibited the lowest stain intensity. However, these lipidized implants still displayed significantly higher stain intensity compared to the sham controls at 31 μm (7.0±0.64; p=9.2×10⁻²⁰). The group that underwent sham surgery exhibits the lowest intensity profile, significantly lower than that observed in the PET and control implant groups (p=5.2×10⁻⁶ and 1.1×10⁻⁵⁶, respectively). The biohybrid model played a significant role in decreasing the deposition of reactive astrocytes on our implants at day 28, specifically when analyzing the first 100 μm of brain tissue in close contact with the implant of the ones with and without neuronal membranes with normalized stain intensity of 5.8±1.3 and 7.2±1.6, respectively; p=4.4×10⁻⁷ (FIG. 10C). As a result, it contributes to minimizing the inflammatory microenvironment that arises from implantation.

CD11b/c was quantified next to examine the extent of the FBR capsule using immunohistochemistry at day 28 (FIG. 10D-E). The stain intensity of CD11b/c is significantly reduced across all implant types, suggesting a lower presence of microglia/macrophages in all four groups in comparison to reactive astrocytes (GFAP upregulation). Implants with neuronal membranes and sham surgery group demonstrate the lowest averaged stain intensities (1.51±0.15 and 1.53±0.2, respectively) across 400 μm thickness of brain tissue, and are not significantly different (p=0.389). However, implants with neuronal membranes display significantly lower stain intensity than PET and control implants (p=2.4×10⁻⁹ and 1.2×10⁻¹⁹, respectively) (FIG. 10D). Analyzing the first 100 μm closest to the device, the lipidized implants display a significantly lower stain intensity (ratio) of 1.7±0.12, compared to control implant at 1.9±0.16 (p=9.5×10⁻²⁰) (FIG. 10E).

The fluorescence images obtained from four experimental groups demonstrate variations in the intensity of staining (FIG. 10F-M). The images were normalized and background fluorescence was corrected for each single image. A comparison between groups that received neuronal membranes as a biohybrid bridge and those that only received a flexible substrate (represented by a single animal where the left and right hemispheres received one lipidized and one control implant) showed an increase in the presence of GFAP and CD11b/c at the edge of the cortical surface for control implants (without neuronal membranes) (FIG. 10B-R). It is important to note that even the initial surgery (sham surgery group) caused a response in both stains, albeit a mild one, while PET caused the highest stain intensity (FIGS. 10B&D).

### Electrophysiology Recordings of Neuronal Membrane-Derived ECoG Arrays In Vivo

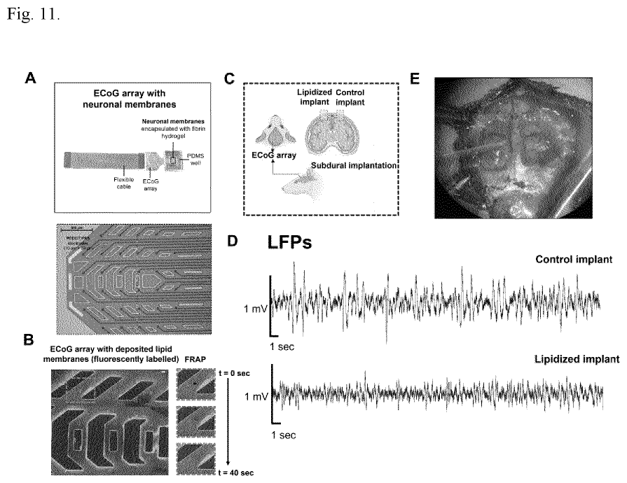

Neural interfaces need to establish connections with surrounding tissue to effectively carry out their intended function, so it was sought to test whether the presence of lipid membranes could potentially hinder the performance of brain recording devices. A ECoG array was created incorporating the NMemb-Biohybrids to record in vivo electrophysiological signals (FIG. 11A, which shows: an image of the device before implantation with a PDMS (Polydimethylsiloxane) well where SLBs and fibrin hydrogel are contained and interfaced on Au/PEDOT:PSS electrodes (top); and ECoG device on PaC substrate with Au-PEDOT:PSS-coated microelectrodes (a total of 32 electrode per device) used for recording LFPs (subdurally) connected to an external cable (brown cable) (bottom)). The array had 32 electrodes, each 30 μm×30 μm in size, and included polygon holes for movement of cerebral spinal fluid (FIG. 11A, bottom). The gold electrodes were coated with PEDOT:PSS, neuronal SLBs, and fibrin hydrogel. The device was placed subdurally on the cortex, and local-field potential (LFP) signals were recorded on the same day of implantation. The ECoG arrays can incorporate neuronal SLB structures within a fibrin hydrogel matrix, or consist solely of the fibrin hydrogel material as a control implant without any neuronal membranes. These arrays can record LFPs from subdural cortical regions with 32 Au/PEDOT:PSS-based electrodes.

Prior to cortical implantation and electrophysiological recordings, FRAP of the ECoG array was conducted to ensure the high mobility and diffusivity of the neuronal membrane on its heterogeneous surface (FIG. 11B, which shows: images of fluorescently labelled membranes (0.25% BODIPY DHPE) on the electronic device pre-implantation where one electrode (pink box) demonstrates mobility of the membrane after photobleaching the defined ROI (dark circle) on the site with a dimeter of 30 μm; images on the right display photobleaching at t=0-40 seconds, demonstrating the recovery in fluorescent intensity overtime, and thus lateral diffusion of the lipid molecules on the ECoG surface; Scale bar=30 μm). The results showed that the neuronal membranes were mobile on both the active sites of the Au/PEDOT:PSS electrodes and the PaC surfaces of the ECoG array, albeit slower on the latter. Due to the smaller microelectrodes, the impedances of these devices are higher than those of previously reported in vitro microelectrodes. When placed subdurally on rats in the lateral cortical parietal areas (FIG. 11C-E), the impedance of the lipidized device is larger than the lipid-free device, confirming the presence of the SLBs on electrodes (16.6±5.8 MΩ and 4.3±3.4 MΩ, respectively). FIG. 11C (created using Biorender.com) shows a schematic of the subdural implantation or brain target, showing both lateral parietal areas between Bregma and Lambda where the implant is interfaced. FIG. 11D shows LFPs measured using two different arrays (control vs. lipidized implants), demonstrating that electrophysiological recordings are successful with the lipidized implant. Note: the devices are recording different areas, hence, showing different LFP traces, N=2, n=4. FIG. 11E shows a captured image of the subdural implantation surgery, showing PEDOT:PSS-based ECoG arrays: one encompassing the neuronal membranes within a fibrin hydrogel, while the other does not. LFP=local-field potential.

Despite the higher impedance, LFP recordings were successfully obtained with the lipidized devices, yielding a comparable SNR compared to the lipid-free device (9.3±3.2 and 10.5±5.4, respectively) (FIG. 11D). It is hypothesized that the presence of transmembrane proteins and the compositional complexity of neuronal membranes contribute to ion movement at the tissue-electrode interface, allowing for similar SNRs despite the heterogeneous membrane asymmetry, thereby accounting for the higher impedance observed.

### Discussion

In the present embodiment, the neuronal membranes demonstrate high electrical sealing properties compared to astrocytes with some defects, likely attributed to the presence of pore-forming proteins or membrane leakiness. Although “lipid-only” membranes are easier to prepare and work with, the iPSC-derived neuronal membranes of the present embodiment present a myriad of discernible advantages in neural interfaces. One key advantage is the potential for functionalization, allowing for additional functionalities to be incorporated into the NMemb-Biohybrids. Functionalization can involve capturing specific transmembrane proteins (i.e., ionotropic glutamate receptors) via cellular blebbing and SLB formation, that are most relevant to neuronal excitability. The versatility enables the creation of tailored membrane interfaces that can modulate cellular responses, improve biocompatibility, and potentially study the electrical properties of host neuronal activity by replicating their physiological conditions. Manipulating the lipid-protein composition is crucial to reduce the high impedance associated with the barrier-forming layer of the membrane, without compromising the device.

In comparison to other anti-fibrotic strategies employed in neural implants, such as surface coatings or drug-delivery systems, neuronal membranes as employed in the present embodiment provide a multi-modal approach. While the impact of mechanical properties on tissue-implant interactions has been extensively studied in peripheral tissues, the investigation of these effects in soft tissues, such as the brain and spinal cord, remains relatively limited. Dexamethasone administration has shown effectiveness in reducing fibrous capsule formation and improving neuroprosthetic implant performance, its broad anti-inflammatory effects can hinder tissue healing, which is critical for regenerative implants. In contrast, the NMemb-Biohybrids of the present embodiment provide a targeted and more of a biologically relevant interface that promotes integration and long-term implant stability in the brain. The implementation of an animal-free membrane extraction protocol in the NMemb-Biohybrids also mitigates non-host immune rejections, expanding its applicability beyond rat models. Surface coatings, such as fibrin-based hydrogel coatings, offer a passive approach compared to the NMemb-Biohybrids, which may actively integrate nanometer-scale components like ion channels at the surface level. This active functional approach can potentially enhance long-term stability and improve recording or stimulation performance in neural interfaces.

Also, the versatility of this embodiment extends beyond the brain, enabling it to be applied to peripheral nerve interfaces and neuromuscular interfaces, replicating the distinct properties of sensory or motor neuronal membranes for the development of specialized interfaces that enhance sensory feedback, motor control, and functional restoration in cases of neural injury or disease. This highlights the increased biocompatibility achieved.

Accordingly, the integration of human iPSC-derived neuronal membranes into neural implants in accordance with the present embodiment represents a promising solution to the long-standing challenge of achieving long-term stability in vivo. By leveraging flexibility of electronics and biocompatibility of biologically complex membranes, this innovative approach is able to mitigate the foreign body response and improve the efficacy and stability of neural interfaces as well as maintain high SNR of electrophysiological recordings, despite lipids insulating nature. These findings open up new avenues exploiting cellular mechanisms to harness in bioengineering for the development of chronic therapeutic interventions and the advancement of biocompatible neural interfaces.

The forgoing description is exemplary in nature only, and the skilled person will understand that changes and variations on the disclosed embodiments are possible within the scope of the claims.

What is claimed is:

1. A medical device, comprising:
a flexible electrode array having a bend radius of no more than about 2 mm; and
a culture of biological cells on the flexible electrode array and electrically integrated with the electrodes of the flexible electrode array.

2. The medical device according to claim 1, wherein said culture of biological cells comprises at least one of myocytes, neurons and fibroblasts.

3. The medical device according to claim 2, wherein said biological cells comprise skeletal myocytes.

4. The medical device according to claim 1, wherein said biological cells are generated from human induced pluripotent stem cells (iPSCs).

5. The medical device according to claim 1, wherein said culture of biological cells is a plurality of biological cell components.

6. The medical device according to claim 5, wherein said biological cell components are cell membranes.

7. The medical device according to claim 6, wherein said cell membranes are neuronal membranes.

8. The medical device of claim 7, wherein said neuronal membranes are human iPSC-derived neuronal membranes.

9. The medical device according to claim 1, wherein the flexible electrode array comprises electrodes provided on a flexible substrate.

10. The medical device according to claim 9, wherein said flexible substrate comprises at least one of a parylene sheet and a polymer sheet comprising parylene.

11. The medical device according to claim 1, wherein said flexible electrode array comprises conducting polymer electrodes.

12. The medical device according to claim 11, wherein said conducting polymer electrodes comprise PEDOT:PSS.

13-25. (canceled)

26. A medical device, comprising:
a flexible electrode array having a bend radius of no more than about 2 mm; and
a plurality of biological cell components on the flexible electrode array and electrically integrated with the electrodes of the flexible electrode array.

27. The medical device according to claim 26, wherein said biological cell components are cell membranes.

28. The medical device according to claim 27, wherein said cell membranes are neuronal membranes.

29. The medical device of claim 28, wherein said neuronal membranes are human iPSC-derived neuronal membranes.

30-33. (canceled)
